(19) United States
(12) Patent Application Publication
(10) Pub. No.: US 2025/0298007 A1
(43) Pub. Date: Sep. 25, 2025

(54) GENETICALLY-ENCODED VOLTAGE SENSOR FOR THE MITOCHONDRIAL INNER MEMBRANE

(71) Applicant: Wisconsin Alumni Research Foundation, Madison, WI (US)

(72) Inventors: Meyer Jackson, Madison, WI (US); Masoud Sepehri Rad, Madison, WI (US)

(73) Assignee: Wisconsin Alumni Research Foundation, Madison, WI (US)

(21) Appl. No.: 18/609,367

(22) Filed: Mar. 19, 2024

(51) Int. Cl. G01N 33/50; G01N 33/531; G01N 33/542

(52) U.S. Cl. CPC G01N 33/5079; G01N 33/5091; G01N 33/531; G01N 33/542

(57) ABSTRACT

As described herein, a hybrid voltage sensor genetically-encoded voltage indicator (GEVI) for mitochondria includes a transmembrane domain portion, and a fluorescent protein, wherein a terminus of the transmembrane domain portion and a terminus of the fluorescent protein are covalently linked directly or by a linker comprising 1 to 20 amino acids, and wherein the transmembrane domain portion comprises two or more copies of SEQ ID NO: 1 or a peptide with greater than 85%, 90%, 95% or 98% identity to SEQ ID NO: 1. Also described are expression vectors, expression cassettes, organelle membranes, and cell lines, as well as methods of determining the voltage across an organelle using the GEVIs.

### STATEMENT REGARDING FEDERALLY SPONSORED RESEARCH & DEVELOPMENT

This invention was made with government support under NS127219 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Mar. 18, 2024 is named “SEQ_LIST—107668285-P240211US01.xml” and is 5,219 bytes in size.

### BACKGROUND

Mitochondria are delimited by membranes with essential roles in their biological functions. These membranes have voltage gradients that can vary and influence how these organelles carry out many of their tasks. Mitochondria have a voltage gradient across their inner membrane that is coupled to the ATP-producing electron transport chain. This voltage is the most important factor in determining mitochondrial capacity for energy generation and constitutes a key index of metabolic health. Mitochondrial dysfunction underlies many diseases, and the role of the mitochondrial membrane in pathological conditions is poorly understood. While voltage at the plasma membrane of a cell can be readily measured, methods of studying the internal electrical signals of mitochondrial membranes are very limited. The inaccessibility of mitochondrial membranes to electrical measurement has resulted in an enormous gap in our understanding of a wide range of normal and pathological cellular processes.

What is needed are novel genetically-encoded plasma membrane voltage indicators (GEVIs) to serve as mitochondrial voltage indicators.

### BRIEF SUMMARY

In an aspect, a hybrid voltage sensor genetically-encoded voltage indicator (GEVI) comprises a transmembrane domain portion, and a fluorescent protein, wherein a terminus of the transmembrane domain portion and a terminus of the fluorescent protein are covalently linked directly or by a linker comprising 1 to 20 amino acids, and wherein the transmembrane domain comprises two or more copies of SEQ ID NO: 1, or a peptide with greater than 85%, 90%, 95% or 98% identity to SEQ ID NO: 1.

In another aspect, an expression vector or an expression cassette comprises a polynucleotide encoding the GEVI described above.

Also included is a stable cell line expressing the GEVI described above.

In a further aspect, an organelle membrane comprises the GEVI, the expression vector comprising a polynucleotide encoding the GEVI, or cassette comprising a polynucleotide encoding the GEVI.

In an aspect, a method of determining the voltage across an organelle membrane comprises expressing the GEVI in the organelle membrane, or delivering the GEVI to the organelle membrane, contacting the organelle membrane with a FRET partner for the fluorescent protein of the GEVI, applying a voltage to the plasma membrane, and recording a voltage change across the organelle membrane by fluorometry of the fluorescent protein-FRET partner.

### BRIEF DESCRIPTION OF THE DRAWINGS

FIG. 1 is a schematic of an embodiment of a hybrid voltage sensor type of GEVI.

FIGS. 2-6. show the inner mitochondrial membrane voltage sensor (4x-mt-mCerulean3). FIG. 2. Top=domain map of the probe. The Cox8A sequence is repeated 4 times with the indicated tripeptide linkers (GDP, GKLAT (SEQ ID NO: 2), GDP) and then linked with a 9 amino acid sequence (SEQ ID NO: 2) to cerulean fluorescent protein (mCerulean3, blue). Bottom=the 29 amino acid sequence (SEQ ID NO: 1) labeled Cox8A in the domain map is from cytochrome oxidase subunit 8A. FIG. 3. A bright field image of a patch-clamped HEK 293 cell expressing the probe (patch electrode visible to the left). FIG. 4. Fluorescence image of the same cell. FIG. 5. Difference image generated by subtracting the fluorescence images before and after the patch clamp stepped the voltage from −70 to −200 mV (averages of 20 frames at the times indicated by the dash lines in FIG. 6.). FIG. 6. Fluorescence versus time from the voltage clamped HEK 293 cell in 2B-D as the patch clamp applies a voltage step indicated by the black trace below. Images were recorded at a frame rate of 200 Hz. The scale bar=10 μm.

The above-described and other features will be appreciated and understood by those skilled in the art from the following detailed description, drawings, and appended claims.

### DETAILED DESCRIPTION

Described herein are hybrid voltage sensors (hVOS), in which membrane targeting functions are clearly delineated from voltage sensing functions. The inventors used hVOS probes as GEVIs to serve as mitochondrial voltage indicators. Preliminary data in cultured cells indicate that these probes target mitochondria with very high efficiency and produce robust fluorescence changes in response to voltage steps. These probes will have wide ranging applications in basic biomedical research, and in the study of disease models. They will be especially useful in screening drugs that target organelle function.

In an aspect, an hVOS type of GEVI comprises a transmembrane domain portion, and a fluorescent protein, wherein a terminus of the transmembrane domain portion and a terminus of the fluorescent protein are covalently linked directly or by a linker, and wherein the transmembrane domain portion comprises two or more copies of SEQ ID NO: 1, or a peptide with greater than 85%, 90%, 95% or 98% identity to SEQ ID NO: 1.

### MSVLTPLLLRGLTGSARRLPVPRAKIHSL (SEQ ID NO: 1)

SEQ ID NO: 1 is a signal peptide of mitochondrial cytochrome oxidase subunit 8 (Cox8). Cox8 couples the transfer of electrons from cytochrome c to molecular oxygen and contributes to a proton electrochemical gradient across the inner mitochondrial membrane.

As used herein, the terms “identical” or percent sequence “identity” in the context of two or more proteins, refer to two or more sequences or subsequences that are the same or have a specified percentage of amino acid residues that are the same, when compared and aligned (introducing gaps, if necessary) for maximum correspondence. The percent identity can be measured using sequence comparison software or algorithms or by visual inspection. Various algorithms and software are known in the art that can be used to obtain alignments of amino acid sequences.

The percent sequence identity “X” of a first amino acid sequence to a second sequence amino acid is calculated as 100 times (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

In an aspect, a sequence with a specified percentage of sequence identity includes conservative amino acid substitutions.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In an aspect, the transmembrane domain comprises only conservative amino acid substitutions.

The transmembrane domain portion of the GEVI comprises two more, such as 2, 3, 4, 5, 6, 7, 8, 9, or 10, preferably four copies of SEQ ID NO: 1.

In an aspect, the two or more copies of SEQ ID NO: 1 that form the transmembrane domain portion are connected by linkers, such as a 1-20, 1-15, 1-5, or specifically 3-5 amino acid linkers. Exemplary linkers include GDP and GKLAT (SEQ ID NO: 2).

A terminus of the transmembrane domain portion and a terminus of the fluorescent protein are covalently linked directly or by a linker. The linker can maintain a certain minimum proximity between the terminus of the transmembrane domain portion and a terminus of the fluorescent protein and ensures efficient energy transfer between the fluorescent protein and fluorescence resonance energy transfer (FRET) partner when they are on the same side of the membrane, even at low concentrations. In an aspect, the linker comprises 1 to 20 amino acids. An exemplary linker to connect the terminus of the transmembrane domain portion and a terminus of the fluorescent protein is GKLRILQST (SEQ ID NO: 3). An exemplary transmembrane domain portion plus linkers has SEQ ID NO: 4.

(SEQ ID NO: 4)
MSVLTPLLLRGLTGSARRLPVPRAKIHSLGDPMSVLTPLLLRGLTGSA
RRLPVPRAKIHSLGKLATMSVLTPLLLRGLTGSARRLPVPRAKIHSLG
DPMSVLTPLLLRGLTGSARRLPVPRAKIHSLGKLILQST

The transmembrane domain portion is covalently linked to a fluorescent protein. In an aspect, the fluorescent protein has an emission maximum between 400 and 550 nm. Exemplary fluorescent proteins include green fluorescent protein (GFP), enhanced GFP (eGFP), farnesylated enhanced GFP (eGFP-F), cerulean fluorescent protein (CeFP), teal fluorescent protein (TeFP), enhanced cyan fluorescent protein (ECFP), enhanced yellow fluorescent protein (EYFP), mTurquoise fluorescent protein, or mTagBFP monomeric blue fluorescent protein.

In an aspect, the GEVI is in electrical communication with a FRET partner for the fluorescent protein. In an aspect, the FRET partner for the fluorescent protein is dipicrylamine (DPA), a (thio)barbiturate oxonol such as DiSBA-C₂, or 4-amino-4′-nitroazobenzene (D3).

In an aspect, the FRET partner has an absorption peak between 350 and 550 nm.

DPA is a nonfluorescent absorber with an absorption maximum of 420 nm, and it has spectral overlap with the emission of fluorescent proteins such as GFP and CeFP. Depending on the membrane potential, DPA molecules can be distributed between the outer and inner faces if a lipid membrane. At a resting membrane potential, the negatively-charged DPA molecules are mostly in the outer face of the membrane, so the emission of the fluorescent protein is unquenched. Upon membrane depolarization, the DPA molecules translate from the outside to the inside of the membrane. The closer proximity enables the DPA to quench the fluorescence of the fluorescent protein by FRET.

The term “polymethine oxonol” refers to molecules comprising two potentially acidic groups linked via a polymethine chain and possessing a single negative charge delocalized between the two acidic groups. The preferred acidic groups are barbiturates or thiobarbiturates. They may be symmetric or asymmetric, i.e., each of the two (thio)barbiturates may be the same or different. The symmetric (thio)barbiturate oxonols are described by the conventional shorthand DiBA-Cₙ-(x) and DiSBA-Cₙ-(x), where DiBA refers to the presence of two barbiturates, DiSBA refers to the presence of two thiobarbiturates, Cₙ represents alkyl substituents having n carbon atoms on the nitrogen atoms of the (thio)barbiturates, and x denotes the number of carbon atoms in the polymethine chain linking the (thio)barbiturates. Exemplary symmetric (thio)barbiturate oxonols include DiSBA-C₂-(3), DiSBA-C₆-(3), DiSBA-C₁₀-(3), DiSBA-C₄-(3).

D3 is 4-amino-4′-nitroazobenzene, also known as Disperse Orange 3.

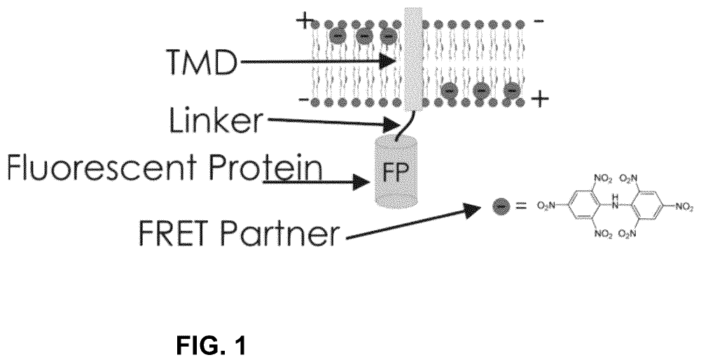

In an aspect, as shown in FIG. 1, the transmembrane domain portion of the GEVI spans the mitochondrial inner or outer membranes or the perimitochondrial space. In a specific aspect, the FRET partner for the fluorescent protein partitions within a mitochondrial membrane.

Advantageously, the GEVI exhibits no measurable fluorescence in a plasma membrane.

Also included herein is an expression vector comprising an expression cassette for the GEVIs described herein.

The terms “expression vector” or “vector” as used herein refers to nucleic acid molecules, typically DNA, to which nucleic acid fragments encoding a GEVI can be propagated. A vector will typically contain one or more unique restriction sites and may be capable of autonomous replication in a defined host cell or vehicle organism such that the cloned sequence is reproducible. A vector may also contain a selection marker, such as, e.g., an antibiotic resistance gene, to allow selection of recipient cells that contain the vector. Vectors may include, without limitation, plasmids, phagemids, bacteriophages, bacteriophage-derived vectors, PAC, BAC, linear nucleic acids, e.g., linear DNA, viral vectors, etc., as appropriate. Expression vectors are generally configured to allow for and/or effect the expression of nucleic acids or ORFs introduced thereto in a desired expression system, e.g., in vitro, in a host cell, host organ and/or host organism. For example, expression vectors may advantageously comprise regulatory sequences.

An expression cassette contains a promoter that starts transcription of a gene, the gene itself, and a transcription termination sequence.

As used herein, the term “promoter” refers to a DNA sequence that enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control expression of a coding sequence of interest, such as the nucleic acid molecule as defined herein.

The promotor may be a constitutive or inducible (conditional) promoter. A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule such as tamoxifen) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues.

In an aspect, a method of determining the voltage across an organelle membrane in a cell comprises expressing the GEVI in the organelle membrane, or delivering the GEVI to the organelle membrane, contacting the organelle membrane with a FRET partner for the fluorescent protein of the GEVI, applying a voltage to the plasma membrane of the cell, and recording a voltage change across the organelle membrane by fluorometry of the fluorescent protein-FRET partner.

An expression vector or expression cassette can be delivered to a specific cell type by a targeted viral vector. Targeted viral vectors can be prepared by pseudotyping (transferring viral attachment proteins), using adaptor proteins (dual specific molecules that bind both a viral attachment protein and a receptor on a target cell), genetic incorporation of targeting ligands, and the like. Adaptor proteins include receptor-ligand complexes, chemically conjugated adaptors, avidin/biotin, camelid nanobodies, and monoclonal antibodies, for example.

Also included is a stable cell line such as a human embryonic kidney cell stably expressing the GEVI. Biological cells include, but are not limited to, primary cultures of mammalian cells, cells dissociated from mammalian tissue, either immediately or after primary culture, or cells in acute tissue preparations such as brain slices. Cell types include, but are not limited to, white blood cells (e.g., leukocytes), hepatocytes, pancreatic beta-cells, neurons, smooth muscle cells, intestinal epithelial cells, cardiac myocytes, glial cells, and the like. Cell types also can include cells derived from human stem cells.

The patch-clamp technique allows one control the voltage across a membrane and to measure the ion currents flowing through the clamped membrane. The patch-clamp fluorometry technique (PCF), an approach combining fluorescence recordings and patch-clamp recordings, permits the simultaneous correlation of ionic current recordings with the activity of protein conformational changes reported by the fluorescence measurement. An exemplary experimental set-up comprises an Olympus BX51 microscope and a CCD-SMQ camera or a DaVinci 2K camera or a Kinetix camera. The CCD-SMQ acquires images at up to 2 kHz with a resolution of 80×80; the DaVinci 2K and Kinetix have higher speed and spatial resolution. A Prizmatix LED UHP—F-HCRI white light source can be used to provide strong and stable excitation in the critical spectral bands and excite multiple probes. A laser with appropriate power and stability can also be used. An assortment of high NA objectives with magnifications ranging from 10× to 60× can be used. Patch clamping can be performed with an Axopatch 200B, a Digidata interface, a computer, and PClamp software. These set-ups can be used to patch clamp cells expressing GEVIs, apply voltage steps, and measure fluorescence responses.

In specific aspects, the GEVIs can be used for drug screening for drugs which affect the membrane potential of organelles, specifically mitochondria. Detection of a change in membrane potential or membrane potential changes in response to the test agent relative to the control indicates that the test agent is active. The control can be a reference drug or no drug. Organelle membrane potentials change in response to a variety of biological challenges. In an aspect, a test agent improves mitochondrial function. Mitochondria respond to metabolic stress resulting from nutrient shortage or oxidative stress or a disruption of metabolism, or in the case of neurons, stress resulting from excessive electrical activity, glutamate, H₂O₂, or an NO donor. A GEVI can monitor these organelle voltage changes in the presence or absence of a test agent to indicate if the test agent has an action on this organelle response. Membrane potentials and membrane potential responses can also be determined in the presence or absence of a pharmacologic agent of known activity (i.e., a standard agent) or putative activity (i.e., a test agent). A difference in membrane potentials or membrane potential responses as detected by the methods disclosed herein allows one to compare the activity of the test agent to that of the standard agent. Mitochondrial stress is a precursor to cell death in a variety of pathological conditions and their dysfunction contributes to neurodegeneration in diseases such as Alzheimer's and Parkinson's disease. A mitochondrial GEVI would be useful in screening drugs that correct mitochondrial dysfunction in the treatment of these diseases.

Biological cells include, but are not limited to, primary cultures of mammalian cells, cells dissociated from mammalian tissue, either immediately or after primary culture, or cells in acute tissue preparations such as brain slices. Cell types include, but are not limited to, white blood cells (e.g., leukocytes), hepatocytes, pancreatic beta-cells, neurons, smooth muscle cells, intestinal epithelial cells, cardiac myocytes, glial cells, and the like. Cell types also can include cells derived from human stem cells. Cell types also include stable cell lines permanently expressing the GEVI.

The screening methods described herein can be made on cells growing in or deposited on solid surfaces or in suspension. A common technique is to use a microtiter plate well wherein the fluorescence measurements are made by commercially available fluorescent plate readers. The methods include high throughput screening in both automated and semiautomated systems such as the Curi Bio Singray system, the Ionoptix C-Pace EM system, and the Hamamatsu kinetic plate imager.

In an aspect, the organelle is mitochondria, and the method further comprises contacting the cell with a test mitochondrial inhibitor, enhancer, or protective agent.

In an aspect, the organelle membrane is in a cell that is a disease model. GEVIs that target mitochondria can be useful to study disease models and screen drugs related to neurological and neurodegenerative disorders (e.g., Alzheimer's Disease (AD), Parkinson's Disease (PD), traumatic brain injury (TBI), multiple sclerosis, muscular dystrophy), cardiomyopathy, cancer, obesity, hematopoietic dysfunction, maintenance of somatic progenitor cells, and the like. In an aspect, the disease is multiple sclerosis, and the cells comprise cerebellar Purkinje cell axons.

In another aspect, a method of determining the voltage across an organelle membrane comprises expressing a fluorescent protein tagged with a motif that binds to a perimitochondrial matrix protein residing in the space between the mitochondrial inner and outer membranes, contacting the organelle membrane with a FRET partner for the fluorescent protein. The FRET partner within the mitochondrial membrane is sufficiently close to the perimitochondrial space to enable FRET changes when the FRET partner moves within the mitochondrial membrane. Applying a voltage to the cell, and recording a voltage change across the organelle membrane by fluorometry of the fluorescent protein-FRET partner. The organelle GEVI reports the transmission of voltage changes from the plasma membrane to internal membranes of organelles.

The invention is further illustrated by the following non-limiting examples.

### EXAMPLES

### Example 1: Development of a Mitochondrial GEVI

The Table summarizes results for a single mitochondrial targeting motif (Cox8 A) and 4 repeats of Cox8A. This illustrates the complex nature of probe performance. Performance cannot be predicted based on sequence, and probes must be individually tested.

TABLE 1
MEMBRANE VOLTAGE MEASUREMENTS
FOR MITOCHONDRIAL CONSTRUCTS

| | | |
|---|---|---|
| Construct | Signal Direction | Signal kinetics |

| | |
|---|---|
| Mito(Cox8)-mCerulean3, | No signal |
| single copy | |

| | | |
|---|---|---|
| Comment: FP targeted | | |
| to the inner membrane | | |
| Mito(Cox8)-mCerulean3, 4 | Upward for | Slow - 1-2 |
| copies, SEQ ID NO: 3 | negative voltage | seconds |
| Comment: FP targeted | | |
| to the inner membrane | | |

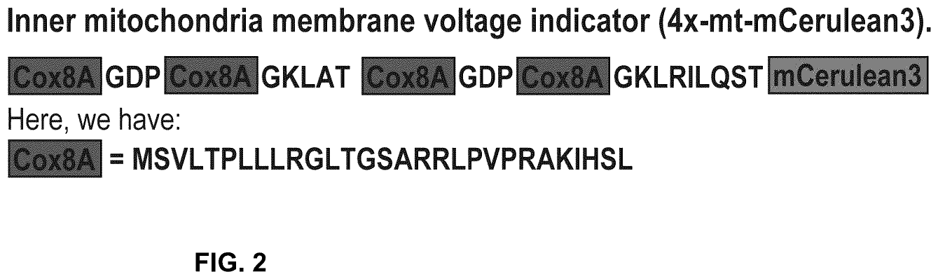

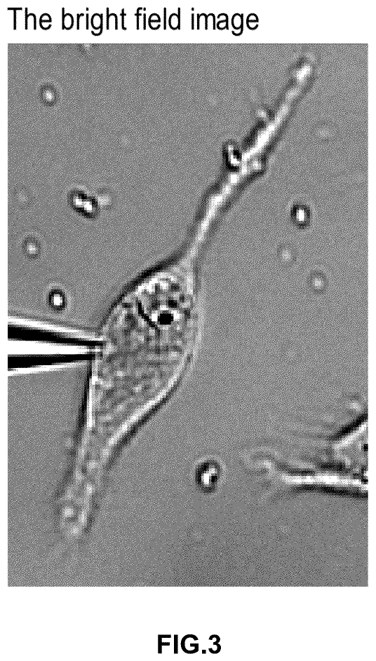

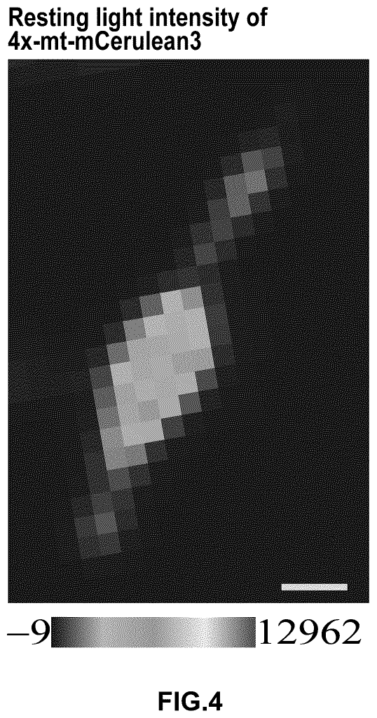

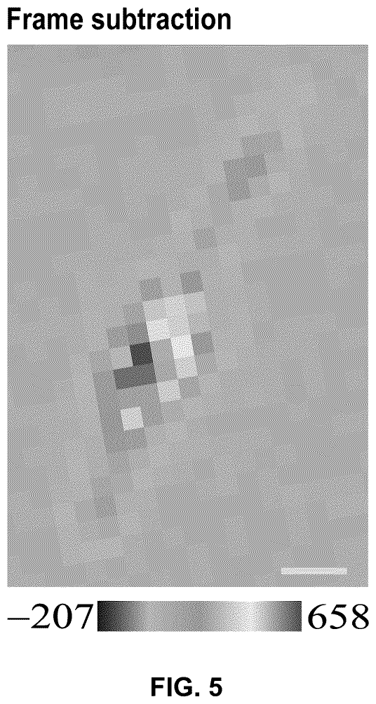

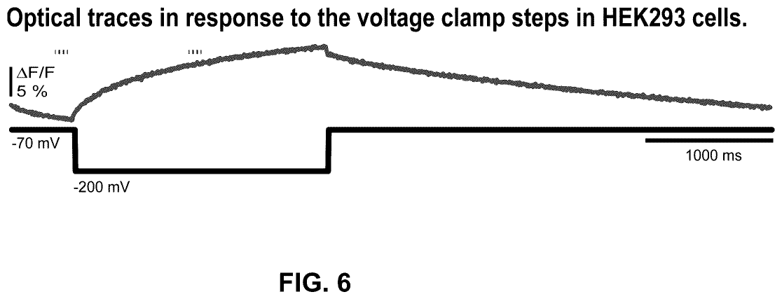

Specifically, as shown previously in U.S. Pat. No. 11,846,628, a probe including a single copy of Mito(Cox8)-mCerulean3 produced no discernable signal. However, unexpectedly, Mito(Cox8)-mCerulean3 with 4 copies, SEQ ID NO: 3, produced a robust signal. FIG. 2 shows the structure and sequence of the Mito(Cox)8 GEVI. FIG. 3 shows a brightfield image of patch-clamped HEK cell. FIG. 4 shows a fluorescence image illustrating internal location of the GEVI. FIG. 5 shows a difference of two images, one before, and the other during a voltage step. This displays the internal cell locations where a voltage change occurs. FIG. 6 shows the fluorescence versus time as the voltage step (indicated below) is applied and the mitochondrial inner membrane changes.

The data in these figures illustrate that Mito(Cox)8 GEVI provides a high quality optical signal that can be readily recorded with standard instrumentation to interrogate the metabolic state of mitochondria in living cells. This probe thus makes it possible to screen drugs and assess experimental manipulations for perturbation of mitochondrial function.

In addition to CeFP, other FPs can be employed. For a variety of tethers, the best FPs can be on the blue-green side of the visible spectrum. CeFP was selected, and a brighter version of CeFP with dramatically improved brightness and/or photostability can be employed. The tethers used for mito-GEVIs can alter the FRET interaction with DPA, so other blue-green FPs with longer and shorter excitation maxima will be tested to determine whether increases or decreases in R₀ for FP-DPA FRET improve voltage sensor performance. For plasma membrane sensing, EGFP, TealFP, and mTurquoise are within 20-30% of CeFP in ΔF/F and they can be tested in organelle-targeted probes. mTagBFP will also be tested because of its high brightness and blue-shifted excitation (peak 402 nm), which should shorten R₀ for FRET with DPA.

Different FRET partners will affect the voltage sensing ability of the GEVIs, so the mito-GEVIs can be tested with two other FRET partners, D3 and DiSBA-C₂-(3). D3 has a peak absorbance at approximately 440 nm, with a shoulder that overlaps substantially with CeFP emission. DiSBA-C₂-(3) is available from ThermoFischer and has an excitation peak at 540 nm and an emission peak at 560 nm, so we will test this FRET partner in a mito-GEVI containing EGFP or YFP. Because DiSBA-C₂-(3) transits the membrane slowly compared to DPA, we will increase pulse duration, and weigh the disadvantage of slower response time against a possible benefit of better signal-to-noise. For each FRET partner we will compare voltage responses, signal-to-noise, and response dynamics. We will also test photostability/phototoxicity in HEK293 cells by repeated data acquisition at 5 min intervals for two hours.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A hybrid voltage sensor genetically-encoded voltage indicator (GEVI), comprising
a transmembrane domain portion, and
a fluorescent protein,
wherein a terminus of the transmembrane domain portion and a terminus of the fluorescent protein are covalently linked directly or by a linker comprising 1 to 20 amino acids, and
wherein the transmembrane domain portion comprises two or more copies of SEQ ID NO: 1, or a peptide with greater than 85% identity to SEQ ID NO: 1.

2. The GEVI of claim 1, wherein the peptide with greater than 85% identity to SEQ ID NO: 1 comprises only conservative amino acid substitutions.

3. The GEVI of claim 1, wherein the transmembrane domain portion comprises four copies of SEQ ID NO: 1 separated by 1-5 amino acid linkers.

4. The GEVI of claim 3, having SEQ ID NO: 4.

5. The GEVI of claim 1, wherein the fluorescent protein has an emission maximum between 400 and 550 nm.

6. The GEVI of claim 1, wherein the fluorescent protein comprises green fluorescent protein (GFP), enhanced GFP (eGFP), farnesylated enhanced GFP (eGFP-F), cerulean fluorescent protein (CeFP), teal fluorescent protein (TeFP), enhanced cyan fluorescent protein (ECFP), enhanced yellow fluorescent protein (EYFP), mTurquoise fluorescent protein, or mTagBFP monomeric blue fluorescent protein.

7. The GEVI of claim 1, in electrical communication with a FRET partner for the fluorescent protein.

8. The GEVI of claim 7, wherein the FRET partner for the fluorescent protein is dipicrylamine (DPA), a (thio)barbiturate oxonol DiSBA-C₂, or 4-amino-4′-nitroazobenzene.

9. The GEVI of claim 7, wherein the transmembrane domain of the GEVI spans the mitochondrial membrane and places the fluorescent protein in the intermembrane space.

10. The GEVI of claim 7, wherein the FRET partner for the fluorescent protein is in communication with a mitochondrial inner membrane.

11. The GEVI of claim 7, wherein the FRET partner has an absorption peak and an emission peak between 350 and 550 nm.

12. An expression vector or an expression cassette comprising a polynucleotide encoding the GEVI of claim 1.

13. A stable cell line such as expressing the GEVI of claim 1.

14. The stable cell line of claim 13 which is a human embryonic kidney cell line.

15. A mitochondrial membrane comprising the GEVI of claim 1.

16. A method of determining the voltage across a mitochondrial membrane, comprising
expressing the GEVI of claim 1 in the mitochondrial membrane, or delivering the GEVI of claim 1 to the mitochondrial membrane,
contacting the mitochondrial membrane with a FRET partner for the fluorescent protein of the GEVI,
applying a voltage to the mitochondrial membrane, and
recording a voltage change across the mitochondrial membrane by fluorometry of the fluorescent protein-FRET partner.

17. The method of claim 16, wherein the method further comprises contacting the cell with a test mitochondrial inhibitor, enhancer, or protective agent.

18. The method of claim 16, wherein the mitochondrial membrane is in a cell that is a disease model.

19. The method of claim 18, wherein the disease model is a model for Alzheimer's Disease (AD), Parkinson's Disease (PD), traumatic brain injury (TBI), multiple sclerosis, muscular dystrophy, cardiomyopathy, cancer, obesity, hematopoietic dysfunction, or maintenance of somatic progenitor cells.

20. The method of claim 19, wherein the disease is multiple sclerosis, and the cells comprise cerebellar Purkinje cell axons.


SEQUENCE LISTING (4 sequences)

SEQ ID NO: 1 - protein, 29 aa, synthetic construct
  source 1..29: note = mitochondrial Cox8 signal peptide
MSVLTPLLLRGLTGSARRLPVPRAKIHSL

SEQ ID NO: 2 - protein, 5 aa, synthetic construct
  source 1..5: note = peptide linker
GKLAT

SEQ ID NO: 3 - protein, 9 aa, synthetic construct
  source 1..9: note = peptide linker
GKLRILQST

SEQ ID NO: 4 - protein, 135 aa, synthetic construct
  source 1..135: note = Cox 8 signal peptide transmembrane domain portion plus linkers
MSVLTPLLLRGLTGSARRLPVPRAKIHSLGDPMSVLTPLLLRGLTGSARRLPVPRAKIHSLGKLATMSVLTPLLLRGLTGSARRLPVPRAKIHSLGDPMSVLTPLLLRGLTGSARRLPVPRAKIHSLGKLILQST